(19) United States
(12) Patent Application Publication
(10) Pub. No.: US 2021/0247397 A1
(43) Pub. Date: Aug. 12, 2021

(54) COMPLEMENT C4D ASSAY

(71) Applicant: SVAR LIFE SCIENCE AB, MALMÖ (SE)

(72) Inventors: Lisbeth WITT, MALMÖ (SE); Yngve SOMMARIN, Malmö (SE); Stefan CARLSÉN, MALMÖ (SE)

(21) Appl. No.: 16/972,319

(22) PCT Filed: Jun. 5, 2019
(86) PCT No.: PCT/EP2019/064620

§ 371 Date: Dec. 4, 2020

(30) Foreign Application Priority Data
Jun. 7, 2018 (EP) 18176524.9

(51) Int. Cl. G01N 33/574; C07K 14/47; C07K 16/18; G01N 33/68

(52) U.S. Cl. CPC G01N 33/57484; C07K 14/472; C07K 16/18; G01N 2333/4716; C07K 2317/20; C07K 2317/33; C07K 2319/00; G01N 33/6893

(57) ABSTRACT

Present invention relates to novel antibodies and their use in an improved method of determining and detecting C4d.

### FIELD OF THE INVENTION

The invention is in the field of medical diagnostics and other medical tools, as well as uses thereof. More in particular, it provides antibodies specifically directed against a component of the complement system, which allows an improved determination of the activation of the classical complement pathway. Even more in particular, the invention provides means and methods for detecting the activation of the classical and lectin pathway. Furthermore, the invention provides means and methods for determining the presence of complement component C4d in a biological sample. Specifically, the invention allows for an improved quantification of C4d.

### BACKGROUND OF THE INVENTION

The complement system is made up of a large number of plasma proteins that react with each other upon an inflammation to help defending the host. This activity was said to complement the activity of antibodies, hence the name. The complement system is a part of the non-specific immune system together with anatomical barriers, phagocytotic cells and chemokines, i.e. the system that provides immediate responses but do not give long-lasting immunity. It is now known that complement is a functional bridge between innate and adaptive immune responses.

The complement system consists of a number of proteins found in the blood, in general synthesized by the liver and phagocytotic cells, and normally circulating as inactive precursors (pro-proteins). When stimulated by one of several triggers, proteases in the system cleave specific complement proteins to anaphylatoxins, activated proteins, and initiate an amplifying cascade of further cleavages. The end-result of this activation cascade is massive amplification of the response and activation of the cell-killing membrane attack complex. Over 30 proteins and protein fragments make up the complement system, including soluble proteins found at high concentrations in blood and cell membrane receptors. They account for about 5% of the globulin fraction of blood serum and can serve as opsonins.

The proteins that constitute the complement system are synthesized mainly by hepatocytes. Significant amounts are also produced by tissue macrophages, blood monocytes, and epithelial cells of the genitourinary tract and gastrointestinal tract.

The complement system can be initiated by three biochemical pathways: the classical pathway, the alternative pathway, and the lectin pathway.

The three pathways of activation all generate analogous proteolytic C3-convertases and the common terminal pathway resulting in membrane attack formation. The classical complement pathway typically requires antigen:antibody complexes (immune complexes) for activation, whereas the alternative and mannose-binding lectin pathways can be activated by spontaneous C3 hydrolysis or carbohydrates respectively, without the presence of antibodies. In all three pathways, C3-convertase cleaves and activates component C3, creating C3a and C3b, and causing a cascade of further cleavage and activation events.

The classical pathway is triggered by activation of the C1-complex. The C1-complex is composed of one molecule of C1q, two molecules of C1 r and two molecules of C1 s, or C1qr²s². The C1 complex is activated when C1q binds to IgM or IgG complexed with antigens. A single IgM can initiate the pathway, while multiple IgGs are needed. This also occurs when C1q binds directly to the surface of the pathogen. Such binding leads to conformational changes in the C1q molecule, which leads to the activation of two C1 r molecules. C1 r is a serine protease. Activated C1 r cleaves C1 s (another serine protease). The C1r²s² component now splits C4 and then C2, producing C4a, C4b, C2a, and C2b. C4b and C2a bind to form the classical pathway C3-convertase (C4b2a complex), which promotes cleavage of C3 into C3a and C3b; C3b later joins with C4b2a (the C3 convertase) to make C5 convertase (C4b2a3b complex).

C4b formed during activation of the classical pathway is then quickly degraded by a serine protease factor I, which in the presence of a cofactor such as C4b-binding protein, generates C4d—the C4 and downstream processes is shared between the classical and Lectin pathway. C4d as a marker for classical pathway has proven ideal since the classical pathway is usually triggered more frequently than the lectin pathway and in particular for autoimmune diseases. Recently, C4d in systemic blood was described as a diagnostic and prognostic marker of lung cancer. It has been widely used as a marker of systemic lupus erythematosus (SLE) and antibody-mediated graft rejection. Although most of the C4d remains bound to the surface of the cell, which originally activated the classical complement pathway, certain portion is released and detectable in blood.

In order to study and understand the role of C4d and the classical pathway activation underlying some diseases there is a need in the art for reagents, typically antibodies, which are specific for C4d. The present application addresses that need.

### SUMMARY OF THE INVENTION

In one aspect, the invention relates to antibodies capable of specifically binding to C4d (anti-C4d). The antibodies may or may not be conjugated with a suitable enzyme such as e.g. Horse Radish Peroxidase (HRP), alkaline phosphatase (ALP), urease or any other suitable enzyme known in the art.

The antibody may be of any origin such as e.g. mammalian, such as e.g. human or mouse etc. In another aspect, the antibody may be avian. The antibody may be monoclonal or polyclonal. The antibodies suitably bind to both allelic variants of C4d, i.e. the A and B chains of C4d. These antibodies are selected to bind both allelic isoforms of C4d, in order to address any homozygotic subjects and to increase the analytical sensitivity in the main population.

In detail, present invention relates to neo-epitope binding antibodies capable of recognizing the newly formed and exposed C-terminus of protein degradation products but fail to recognize or bind to the same sequence of amino acids present in intact or undigested protein form. Consequently, these antibodies may be denoted as anti-C4d-neo.

The antibodies according to the invention may be of any origin such as e.g. mammalian, such as e.g. human, mouse, or of avian origin etc. The antibody may be monoclonal or polyclonal. The above described antibodies may be described as capture antibodies, and in the case of monoclonal antibodies, these may be denoted as capture mAbs.

Moreover, in one aspect of the invention, the invention also relates to recombinant human C4d. Suitably, this is used as a standard and/or control when performing in assay using the above mentioned antibodies.

In a further aspect, the invention relates to an assay for quantification of C4d. The quantification may be performed by employing the herein mentioned antibodies and the recombinant human C4d as standard and/or control.

The assay may be used for any suitable purpose wherein C4d is capable of acting as a biomarker. Increased levels of C4d are associated with many pathological conditions such as e.g. SLE (serum level, deposition on platelets and erythrocytes), NSCLC (non-small cell lung cancer)—plasma and bronchioaveolar levels, antibody-mediated graft rejection (predictive in biopsies of transplants), thrombotic microangiopathy, lupus nephritis, myelopathy due to human leukaemia T-cell virus (HLTV) etc. Generally, C4d is associated with a variety of tumours and autoimmune diseases.

Other applications of the invention as described herein may also be for e.g. histological purposes. In such applications, the antibody according to the invention may be unconjugated to a fluorophore or any other entity. In order to detect such bound antibody, a further antibody (detection antibody) is added capable of binding to the Fc region of the antibody according to the invention, wherein the detection antibody is conjugated to an enzyme as specified herein and detection is enabled by adding a substrate digestible by the conjugated enzyme. In another example, the antibody according to the invention may be directly conjugated to an enzyme and detection is made in accordance with standard ELISA techniques as described herein.

In one aspect, the invention relates to both soluble C4d and/or C4d attached to a surface, such as e.g. attached on a cell surface. In a specific aspect, the invention relates to soluble C4d.

In another aspect, the invention relates to use of one or more antibodies as described herein in a diagnostic method or for histological purposes.

In yet a further method, the invention relates to use of one or more antibodies as described herein in a kit or a kit of parts.

In another aspect, the invention relates to use of one or more antibodies as described herein for quantification of C4d.

In yet a further aspect, the invention relates to a kit or a kit of parts comprising one or more containers such that said containers have a surface coated with one or more of the antibodies as described herein.

The one or more antibodies present in a kit according to the invention may independently of each other be conjugated with a suitable enzyme, such as e.g. e.g. Horse Radish Peroxidase (HRP), alkaline phosphatase (ALP), urease or any other suitable enzyme known in the art. Other entities that may be conjugated to the antibody are e.g. other enzymes, radioactive element or fluorophores. Further non-limiting examples of enzymes may be β-galactosidase, acetylcholinesterase and catalase and the likes.

Furthermore, the kit may comprise a suitable standard and/or control. The standard and/or control may be human C4d, such as e.g. recombinant human C4d.

The kit may additionally and optionally comprise a suitable substrate. The substrate may be any substrate devised such that the conjugated enzyme is capable of digesting the substrate. The substrate comprises at least an entity such that once the substrate is digested by the enzyme, detection by any suitable means becomes possible and as a consequence thereof, quantification of C4d is enabled.

Present invention also relates to a method of determining the quantity of C4d.

Specifically, the method of determining the quantity of C4d in a biological sample, may comprise the use of one or more antibodies as described herein and detailed below. Typically, the methods according to the invention may employ the various kits or kit of part as detailed in the application text.

The method may comprise the use of a suitable control. As a control or internal standard, e.g. C4d itself may be used. Preferably, the C4d used as control or internal standard is human and even more preferably, the human C4d is recombinant.

The kit may optionally further comprise a stop solution or agent. The intended use of a stop solution is to stop the reaction used in the assay. The use of a stop solution allows the user to either directly perform the read-out/detection of the signal or may dispense with the need of directly attending to the read out and postpone such action until the stop solution is added. As mentioned in this passage, the stop solution is optional and depending on the substrate used. In some cases, the stop solution may be dispensed with altogether. One non-limiting stop solution or agent may be e.g. sulphuric acid.

### DESCRIPTION OF FIGURES

FIG. 1 illustrates one example of the assay set-up according to the invention and in more detail illustrates a direct sandwich ELISA. The flag represents a reporter that may be an enzyme, a fluorophore or a radioactive element.

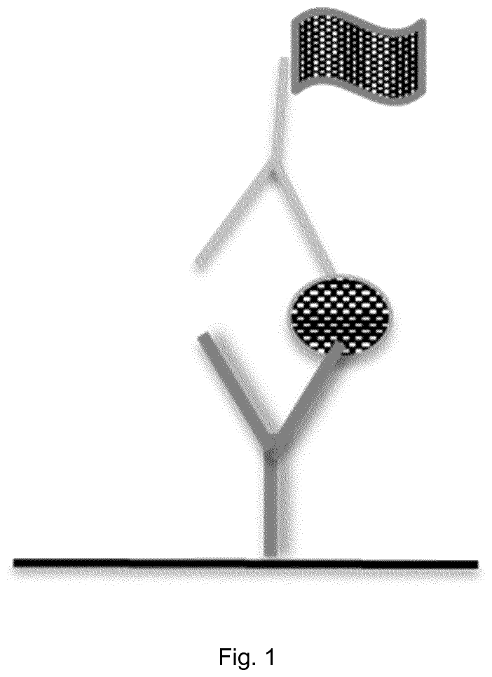

FIG. 2 illustrates the complement C4d assay according to the invention displaying the specificity to complement factor C4d with no cross reactivity to C4, C4b, C3, C3b, C3d, C5, SC5b-C9 or C9 at physiological concentrations. The illustrated assay according to the invention binds both A and B variant of the C4d protein resulting in a high analytical sensitivity.

FIG. 3 illustrates part of the C4d cleaved amino acid sequence from amino acid residue 957 to 1336 and shows both allelic types (A- and B-type) which are illustrated in pairs. The neoepitope is seen as the C-terminal and marked in bold-faced style only. The variable regions/amino acids are marked in bold-faced and underlined style. The A-type is seen in SEQ ID NO.: 2 and the B-type is seen in SEQ ID NO.: 3.

### DETAILED DESCRIPTION OF THE INVENTION

Present invention provides for a higher specificity in binding to C4d and/or higher sensitivity in determining the quantity of C4d in a biological sample. It is estimated that about 30% of the population in the western world have only either the A or B variant of C4d. Thus, present invention presents a major improvement of prior art methods in that both variants are detectable.

In one aspect, the invention relates to an epitope and specifically neo-epitope comprising the amino acid sequence comprising NVTLSSTGR (SEQ ID NO.: 1). Moreover, the invention also relates to an amino acid sequence comprising a sequence with at least 70% sequence identity as set forth in SEQ ID NO.: 1, such as e.g. such as e.g. at least about 75% sequence identity, such as e.g. at least about 80% sequence identity, such as e.g. at least about 85% sequence identity, such as e.g. at least about 90% sequence identity, such as e.g. at least about 95% sequence identity, such as e.g. at least about 98% sequence identity, such as e.g. at least about 99% sequence identity to SEQ ID NO.: 1 or an amino acid sequence identical to SEQ ID NO.: 1. In one aspect, the invention relates to a neo-epitope sequence comprising or consisting of peptide sequence SEQ ID NO.:1 or a peptide sequence having at least about 90% sequence identity, such as e.g. at least about 95% sequence identity, such as e.g. at least about 96% sequence identity, such as e.g. at least about 97% sequence identity, such as e.g. at least about 99% sequence identity to SEQ ID NO.: 1.

In another aspect, the invention relates to antibodies capable of specifically binding to C4d (anti-C4d). Specifically, the antibody is capable of recognising and binding to the amino acid comprising sequence NVTLSSTGR (SEQ ID NO.: 1). Thus an antibody according to the invention is capable of recognising SEQ ID NO.: 1 or a sequence with at least 70% sequence identity as set forth in SEQ ID NO.: 1, such as e.g. such as e.g. at least about 75% sequence identity, such as e.g. at least about 80% sequence identity, such as e.g. at least about 85% sequence identity, such as e.g. at least about 90% sequence identity, such as e.g. at least about 95% sequence identity, such as e.g. at least about 98% sequence identity, such as e.g. at least about 99% sequence identity to SEQ ID NO.: 1 or an amino acid sequence identical to SEQ ID NO.: 1.

As mentioned above, the antibodies according to the invention are capable of binding to and recognising the neo-epitope, but do not recognise a non-cleaved amino acid sequence comprising SEQ ID NO.: 1, wherein SEQ ID NO.: 1 is embedded and thus not being the C-terminal of the peptide sequence. Thus the neo-epitope is meant to be may be the C-terminal, wherein SEQ ID NO.: 1 is the amino acid sequence being the C-terminal end of the peptide sequence. Expressed differently, the sequence NVTLSSTGR may thus either be the sequence or part of a sequence wherein said sequence is the C-terminal and thus the sequence may be NVTLSSTGR(-COON).

The anti-C4d antibody or antibodies according to the invention may be of any origin, such as e.g. any animal or human origin. Preferably, the antibody is of human origin. The antibody may be polyclonal or monoclonal. Preferably, the antibody is monoclonal and even more preferably the antibody may be a monoclonal human antibody. In another embodiment, the antibody may be of a murine origin and specifically may be e.g. a mouse antibody. In a further embodiment, the antibody may be of avian origin.

In a further aspect of the invention, the antibodies of the invention may or may not be natural or non-natural origin. The antibodies may be artificial in the sense that they do not exist in any natural organisms.

As mentioned above, the invention also relates to an antibody capable of specifically binding to both allelic isoforms of C4d, i.e. both the A and the B chain of C4d.

Thus in one aspect, the invention relates to an antibody capable of recognising SEQ ID NO.: 1 or a sequence with at least 70% sequence identity as set forth in SEQ ID NO.: 1, such as e.g. such as e.g. at least about 75% sequence identity, such as e.g. at least about 80% sequence identity, such as e.g. at least about 85% sequence identity, such as e.g. at least about 90% sequence identity, such as e.g. at least about 95% sequence identity, such as e.g. at least about 96% sequence identity, such as e.g. at least about 97% sequence identity, such as e.g. at least about 98% sequence identity, such as e.g. at least about 99% sequence identity to SEQ ID NO.: 1 or an amino acid sequence identical to SEQ ID NO.: 1, and a different antibody capable of recognising and specifically binding to both allelic isoforms of C4d, i.e. both the A and the B chain of C4d.

The antibody capable of binding to both allelic isoforms of C4d is capable of binding to the conserved regions in both the A and B-chains, i.e. the regions that are contain identical amino acid sequences in the A and B-chain of C4d. Thus in one aspect, the antibody capable of binding to both the A and B-chains, are capable of binding to or recognising SEQ ID NO.: 2 and/or SEQ ID NO.: 3 or a sequence with at least 70% sequence identity as set forth in SEQ ID NO.: 2 and/or SEQ ID NO.: 3, such as e.g. such as e.g. at least about 75% sequence identity, such as e.g. at least about 80% sequence identity, such as e.g. at least about 85% sequence identity, such as e.g. at least about 90% sequence identity, such as e.g. at least about 95% sequence identity, such as e.g. at least about 96% sequence identity, such as e.g. at least about 97% sequence identity, such as e.g. at least about 98% sequence identity, such as e.g. at least about 99% sequence identity to SEQ ID NO.: 1 or an amino acid sequence identical to SEQ ID NO.: 2 and/or SEQ ID NO.: 3.

In one aspect, the antibody binding to the neotope may be designated as a capture antibody. The antibody binding to both allelic isoforms of C4d, i.e. both the A and the B chain of C4d may be designated as the detection antibody.

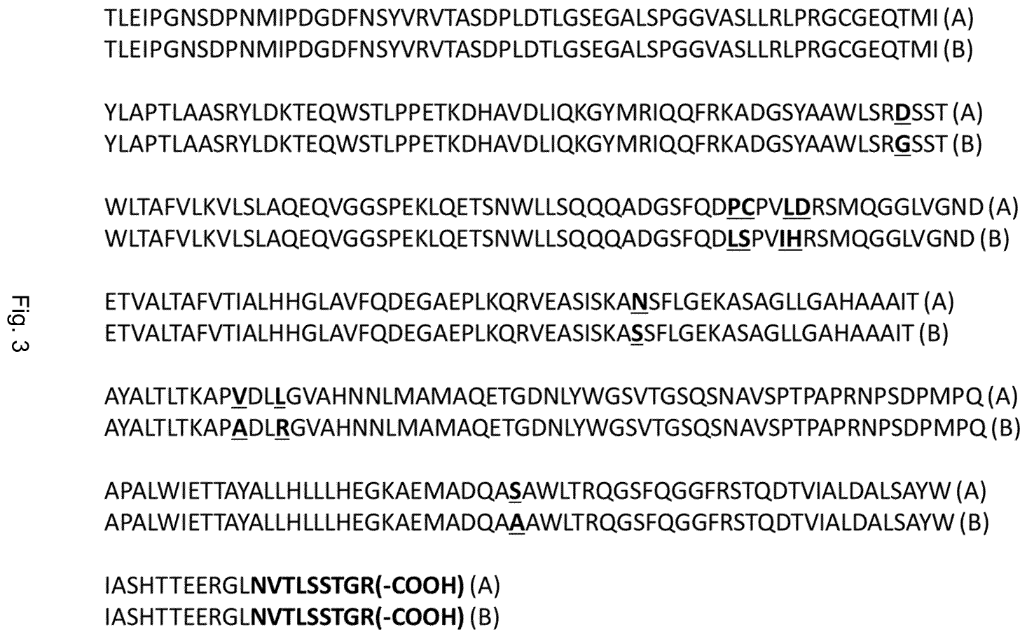

As mentioned above, the detection antibody is capable of binding to both the A- and B-chain of C4d and only to the amino acid sequences that are identical in both chains. The relevant amino acid sequences are in FIG. 3. Specifically, it is seen in FIG. 3 that the amino acid residues that are bold faced and underlined are different between the A and B-chain and consequently only the regions in which the amino acid sequences are identical between the A and B-chain are the relevant amino acid sequences to which a detection antibody is capable to bind. Alternatively to the above, the antibody should be able to recognise an amino acid sequence with at least 4 residues, such as e.g. at least 5 residues, such as e.g. at least 6 residues, such as e.g. at least 7 residues, such as e.g. at least 8 residues, such as e.g. at least 9 residues, such as e.g. at least 10 residues of the regions/amino acid sequences seen in FIG. 3 and which are conserved between the A- and the B-type. It is to be noted that the amino acid residues need not be contiguous.

In one aspect, the invention relates to the use of an antibody capable of recognising SEQ ID NO.: 1 or a sequence with at least 70% sequence identity as set forth in SEQ ID NO.: 1, such as e.g. such as e.g. at least about 75% sequence identity, such as e.g. at least about 80% sequence identity, such as e.g. at least about 85% sequence identity, such as e.g. at least about 90% sequence identity, such as e.g. at least about 95% sequence identity, such as e.g. at least about 98% sequence identity, such as e.g. at least about 99% sequence identity to SEQ ID NO.: 1 or an amino acid sequence identical to SEQ ID NO.: 1, and a different antibody capable of recognising and specifically binding to both allelic isoforms of C4d, i.e. both the A and the B chain of C4d in a diagnostic method. In a further aspect, the antibodies may be used in an assay. In yet a further aspect, the antibodies according to the invention may be used in a so-called sandwich assay.

As mentioned above the invention relates to use of the antibodies according to the invention in an assay or other diagnostic method. In principle, the assay may be any type of assay known in the art. One type of assay may be an ELISA (Enzyme-Linked Immunosorbent Assay) type of assay. The ELISA assay may be of any type known in the art such as e.g. indirect ELISA, sandwich ELISA, or competitive ELISA. Another example of an assay is radioimmunoassay (RIA) which are exemplified below in a non-limiting context.

In one aspect, the one or more antibodies according to the invention may in principle be for any suitable use, such as diagnostic use or for screening purposes. With respect to screening or diagnostic purposes, the antibodies according to the invention may be used for measurement of complement system activation. In another aspect, the antibodies according to the invention may be used for screening of drug candidates or lead compounds in order to investigate the extent with which such drugs trigger the complement system.

### Radioimmunoassay (RIA)

In one aspect, the invention relates to an antibody for use in a RIA. The antibody is capable of binding to an amino acid sequence comprising the amino acid sequences as disclosed herein. In such assay, the assay may further comprise a radioactively labelled target to which the antibody is capable of binding. In one aspect the target may be radioactively labelled C4d. Moreover, in the use of the antibody, the C4d present in the patient sample will competitively bind to the antibody of the invention and the released radioactive C4d will be measured. It is to be understood that the C4d in the patient sample is the neo-epitope relating to an amino acid sequence comprising SEQ ID NO.: 1. The antibody may or may not be immobilised on a solid support such as the wall of a well or any surface.

For example, the antibody of present invention may be mixed with the radiolabelled target such as e.g. radiolabelled C4d. The available binding sites of the antibody will thus be saturated with bound radiolabelled target. Upon contacting the antibody bound to the radiolabelled target (C4d) with a biological sample, the non-radiolabelled target competitively binds to the antibody after which measurement of the released radiolabelled target is performed, allowing for a quantitative measurement.

### Indirect ELISA

In a further aspect, the antibody according to the invention is not conjugated with a suitable enzyme as described herein. Such antibody may be denoted as a primary antibody. The primary antibody is capable of binding to an amino acid sequence comprising SEQ ID NO.: 1.

In such instance the primary antibody is not conjugated with an enzyme, the invention further comprises an antibody capable of recognising the Fc part of the primary antibody. Such antibody may be denoted as a secondary antibody. The secondary antibody may optionally be conjugated with a suitable enzyme, such as e.g. e.g. Horse Radish Peroxidase (HRP), alkaline phosphatase (ALP), urease or any other suitable enzyme known in the art.

In order to allow for detection and ultimately quantification, a substrate capable of being digested by the enzyme conjugated to the secondary antibody is added. The substrate may be any suitable substrate which is capable of being digested by the conjugated enzyme. Non-limiting examples of such substrates are e.g. tetramethyl benzidine (TMB), 2,2′-Azinobis [3-ethylbenzothiazoline-6-sulfonic acid]-diammonium salt (ABTS), p-Nitrophenyl Phosphate, Disodium Salt (PNPP), o-phenylenediamine dihydrochloride (OPD) etc.

### Sandwich ELISA

The invention also relates to a detection antibody capable of specifically binding to C4d at a site different from the neo-epitope described herein (i.e. sites having sequences different from amino acid sequence SEQ ID NO.: 1). Consequently, the detection antibody is different from the antibody according to the invention which is capable of binding to amino acid sequence SEQ ID NO.: 1 and consequently binds to a different epitope of the antigen such as e.g. C4d, which in this example is the A and the B-chain of C4d. In this context, the antibody according to the invention capable of binding to SEQ ID NO.: 1 is denoted as the capture antibody. The antibody capable of binding to the A and B-chain of C4d may be denoted as the detection antibody and thus capable of binding to SEQ ID NO.: 2 and/or SEQ ID NO.: 3.

In order to enable detection and ultimately quantification of the bound antigen, a secondary antibody may be added. The secondary antibody is capable of recognising the Fc part of the detection antibody. The secondary antibody may optionally be conjugated with a suitable enzyme, such as e.g. e.g. Horse Radish Peroxidase (HRP), alkaline phosphatase (ALP), urease or any other suitable enzyme known in the art.

In order to allow for detection and ultimately quantification, a substrate capable of being digested by the conjugated enzyme is added. The substrate may be any suitable substrate which is capable of being digested by the conjugated enzyme. Non-limiting examples of such substrates are e.g. tetramethyl benzidine (TMB), 2,2′-Azinobis [3-ethylbenzothiazoline-6-sulfonic acid]-diammonium salt (ABTS), p-Nitrophenyl Phosphate, Disodium Salt (PNPP), o-phenylenediamine dihydrochloride (OPD) etc.

In another aspect of the invention, the detection antibody, may be the antibody according to the invention capable of binding to amino acid sequence SEQ ID NO.: 1. In such instance, the capture antibody, is an antibody capable of specifically binding to the antigen in question having an epitope different from SEQ ID NO.: 1. This concept thus represents a reverse version of the above. In accordance with this aspect, the secondary antibody mentioned above is capable of recognising the Fc part of the antibody which recognises and bind to SEQ ID NO.: 1.

### Competitive ELISA

In one aspect, the invention relates to an antibody capable of binding to the antigen of a biological sample, wherein the antigen comprises sequence SEQ ID NO.: 1. This antibody may in this case be denoted as the primary antibody.

In this aspect the invention further relates to an antibody capable of binding to the Fc part of the primary antibody. This antibody may thus be suitable denoted as the secondary antibody. The secondary antibody may optionally be conjugated with a suitable enzyme, such as e.g. e.g. Horse Radish Peroxidase (HRP), alkaline phosphatase (ALP), urease or any other suitable enzyme known in the art.

In order to allow for detection and ultimately quantification, a substrate capable of being digested by the conjugated enzyme is added. The substrate may be any suitable substrate which is capable of being digested by the conjugated enzyme. Non-limiting examples of such substrates are e.g. tetramethyl benzidine (TMB), 2,2′-Azinobis [3-ethylbenzothiazoline-6-sulfonic acid]-diammonium salt (ABTS), p-Nitrophenyl Phosphate, Disodium Salt (PNPP), o-phenylenediamine dihydrochloride (OPD) etc.

In order to permit detection and quantification, the primary antibody is incubated with the antigen from a biological sample. The primary antibody bound to its antigen from the biological sample is then added to a surface which may typically by the surface of any type of container. The surface is pre-coated with the same antigen. Any unbound primary antibody (i.e. not bound to said surface), is typically removed by any suitable method such as e.g. rinsing. Said surface is then contacted with the secondary antibody, which ten binds to the Fc part of the primary antibody bound to said surface. A suitable substrate is then added which upon digestion of the enzyme conjugated to the secondary antibody allows for detection and quantification.

Overall and as mentioned herein, the relevant antibodies mentioned throughout the application text may or may not be conjugated to a suitable enzyme. Suitable enzymes are known in the art and exemplified throughout the application text. In performing the assays mentioned herein, a suitable substrate is added for the purpose of detection and quantification. Several suitable substrates are known in the art and consequently, the methods and kits described herein may or may not comprise a substrate which, upon digestion, results in a chromogenic or fluorescent signal which further allows for detection and/or quantification.

The invention also relates to C4d itself, which may be the wild-type C4d. Specifically, the invention further relates to recombinant C4d, and more specifically human recombinant C4d. In the context of the invention, the human recombinant C4d may be used as a control and/or standard. One non-limiting example may be e.g. P0C0L5[957-1336].

### RIA

Present invention also relates to a method of quantifying the presence of an antigen. The antigen may in principle be any antigen such as e.g. C4d in a biological sample.

Consequently, a method according to the invention may comprise:

a) providing an antibody according to the invention, wherein said antibody is pre-loaded with radiolabelled antigen
b) contacting the antibody bound to radiolabelled antigen in a) with a patient sample,
c) measuring the released radiolabelled antigen

The antibody may be an antibody capable of binding to an amino acid sequence comprising SEQ ID NO.: 1, wherein the epitope comprises SEQ ID NO.: 1

### Indirect ELISA

Alternatively, the method may comprise:

a) providing a biological sample from a subject,
b) contacting the sample with the primary antibody according to the invention,
c) adding to the sample a secondary antibody according to the invention,
d) adding to the sample a suitable substrate, and
e) measuring the output of the digested substrate.

The output may be a chromogenic or fluorescent signal.

### Sandwich ELISA

A further alternative, the method according to the invention may comprise the steps of:

a) providing a biological sample from a subject,
b) contacting the sample with the capture antibody according to the invention,
c) adding to the sample a primary antibody according to the invention,
d) adding to the sample a secondary antibody according to the invention,
e) adding to the sample a suitable substrate, and
f) measuring the output of the digested substrate.

The output may be a chromogenic or fluorescent signal.

### Competitive ELISA

In yet a further alternative, the method according to the invention may comprise the steps of:

a) Contacting the primary antibody according to the invention with a biological sample containing an antigen,
b) contacting the primary antibody-antigen complex from step a) with a surface which is pre-coated with the same antigen as in a),
c) removing any unbound antibody in step b),
d) adding a secondary antibody to said surface,
e) adding a substrate, and
f) measuring the output of the digested substrate.

The output may be a chromogenic or fluorescent signal.

It is to be understood that the methods described above may comprise rinsing steps in between any of the steps in the methods. For example, a rinsing step may suitably take place between e.g. step b) and c). In the methods described herein a stop solution may optionally be added in order to stop the enzymatic reaction. This may be added in a step before measuring the readout signal.

Overall, it is also to be understood that the invention may comprise any substrate enabling any form of detection known in the art. For example, the substrate may be used for detection by absorbance or fluorescence or by electrochemical signal in order to quantify C4d. In other aspects, the read-out may be by measuring the radioactivity of the sample.

The method may comprise the use of a suitable control and/or standard. As a control or internal standard, C4d itself may be used. Preferably, the C4d used as control or internal standard is human and even more preferably, the human C4d is recombinant. The measurement/quantification of C4d is usually made by dividing the detection signal from the sample with the signal from the control or internal standard or alternatively the signal from the sample is compared in relation to the signal of the control or internal standard.

Present invention also relates to a kit or a kit of parts. The kit may comprise a one or more antibodies as described herein optionally conjugated with a suitable enzyme, such as e.g. e.g. Horse Radish Peroxidase (HRP), alkaline phosphatase (ALP), urease or any other suitable enzyme known in the art. The one or more antibodies may be the primary or detection antibodies as described herein. These antibodies may optionally be conjugated to a suitable enzyme.

The kit may further comprise a secondary antibody as described herein optionally conjugated with a suitable enzyme, such as e.g. e.g. Horse Radish Peroxidase (HRP), alkaline phosphatase (ALP), urease or any other suitable enzyme known in the art.

The kit may further comprise a capture antibody capable of specifically binding to the antigen (such as e.g. C4d) at a site different from the neo-epitope described herein (i.e. sites different from amino acid sequence SEQ ID NO.: 1). The capture antibody is different from the primary antibody. In some aspects, the capture antibody is in solution. In a preferred aspect, the capture antibody is directly or indirectly attached to a surface of any kind. For example, the capture antibody may be attached to the surface of a well, such as e.g. the well of a microtiter plate or any format.

The kit may further comprise further comprise a suitable substrate. The substrate may be any substrate capable of being digested by the enzyme conjugated to the relevant antibody as the case may be.

The kit may also comprise a control and/or internal standard.

The kit may be in any suitable form, such as comprising one or more containers. For example, the kit may comprise a microtiter plate of any suitable format. The one or more containers may be coated with the capture antibodies according to the invention on one or more surfaces of the one or more containers. In one aspect, the containers may also be coated with the antigen capable of being recognised by the relevant (primary) antibody.

Specifically, a kit or kit of parts according to the invention may be designed to suite the desired context for the assay and its methodology, such as high through-put screening etc.

### Kit for RIA

In one aspect the kit according to the invention may comprise:

a) an antibody according to the invention capable of binding an antigen comprising SEQ ID NO.: 1,
b) radiolabelled antigen to which the antibody according to a) is capable of binding.

The antigen may be e.g. C4d.

### Kit for Indirect ELISA

In one aspect of the invention, the kit according to the invention may comprise:

a) an antibody according to the invention capable of binding an antigen comprising SEQ ID NO.: 1,
b) an antibody optionally conjugated to an enzyme, capable of binding to the Fc-part of the antibody in a),
c) optionally a substrate which is digestible by the enzyme in b).

### Kit for Sandwich ELISA

In a further aspect of the invention, the kit according to the invention may comprise:

a) an antibody according to the invention capable of binding an antigen comprising SEQ ID NO.: 1,
b) an antibody capable of binding to the same antigen in a), but not binding to SEQ ID NO.: 1,
c) an antibody optionally conjugated to an enzyme capable of binding to the Fc part of the antibody in b),
d) optionally a substrate which is digestible by the enzyme in c).

### Kit for Competitive ELISA

In yet a further aspect of the invention, the kit according to the invention may comprise:

a) an antibody according to the invention capable of binding an antigen comprising SEQ ID NO.: 1,
b) a surface pre-coated with the antigen in a),
c) an antibody optionally conjugated to an enzyme capable of binding to the Fc part of the antibody in a),
d) optionally a substrate which is digestible by the enzyme in c).

The methods, kits and/or antibodies according to the invention may be for use in any diagnostic or histological setting or context. Consequently, the invention relates to the detection of presence or absence of C4d in a biological sample. Importantly, the invention relates to the quantification of C4d in a biological sample. The presence or absence of C4d, or as the case may be, the quantity of C4d may enable the presence or absence of any disease associated with C4d. It may also be used to determine the progression or stage of any diseases associated with C4d. Alternatively, the methods, kit and/or antibodies according to the invention may be used to monitor a treatment method of a subject in need thereof. As a consequence thereof the invention may enable the selection of a suitable treatment of a subject.

Diseases or conditions associated with C4d may be e.g. any condition associated with tumours and autoimmune diseases or more specifically, SLE (serum level, deposition on platelets and erythrocytes), glomerulonephritis, NSCLC (non-small cell lung cancer)—plasma and bronchioaveolar levels, antibody-mediated graft rejection (predictive in biopsies of transplants), thrombotic microangiopathy, lupus nephritis, systemic lupus erythematosus, myelopathy due to human leukemia T-cell virus (HLTV) etc.

### Examples

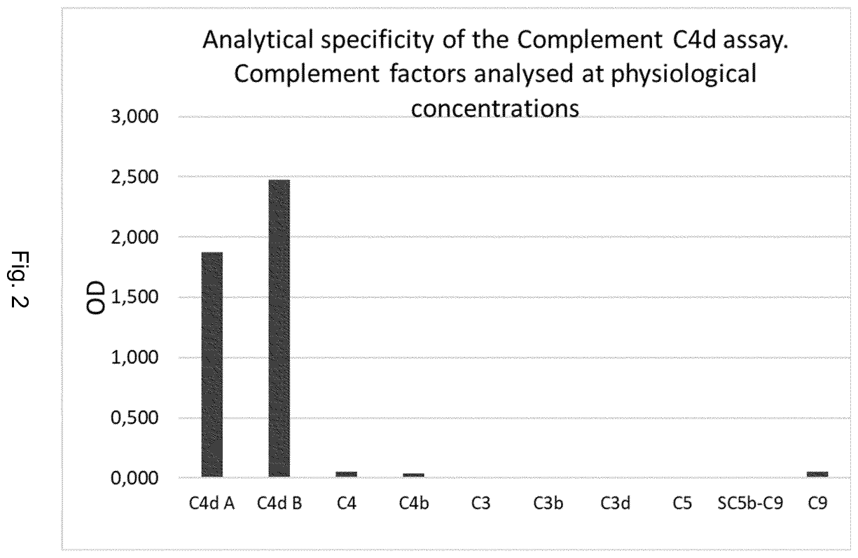

A test was performed to analyze the analytical specificity of the complement C4d assay, as the evolutionary genetically related proteins C3, C4 and C5 and the derivatives thereof can have structural similarities. Analytical specificity was confirmed by analyzing complement factors: C4, C4b, C3, C3b, C3d, C5, SC5b-9 and C9. To challenge cross-reactivity the antigens were analyzed in the ELISA assay at concentrations just above physiological concentrations. The analyzed complement factors gave no signal in the C4d assay (FIG. 2). The test was performed according to the procedure described in the instructions for use.

The ability of the ELISA assay to detect both exciting variants of C4d (A and B) was analyzed. Individuals may lack either C4A, or C4B gene. Partial deficiency of C4A or C4B is the most commonly inherited immune deficiency known in humans with a combined frequency over 31% in the normal Caucasian population. Hence, the analytical sensitivity was tested for both variants to determine binding efficacy of the assay for the total population.

In one aspect the invention relates to the following items:

### Items

1. A polypeptide sequence comprising the SEQ ID NO.: 1, wherein SEQ ID NO.: 1 is the C-terminal and/or N-terminal of a peptide sequence.
2. The polypeptide sequence according to item 1, wherein the polypeptide sequence comprises a sequence with at least 70% sequence identity as set forth in SEQ ID NO.: 1, such as e.g. such as e.g. at least about 75% sequence identity, such as e.g. at least about 80% sequence identity, such as e.g. at least about 85% sequence identity, such as e.g. at least about 90% sequence identity, such as e.g. at least about 95% sequence identity, such as e.g. at least about 98% sequence identity, such as e.g. at least about 99% sequence identity to SEQ ID NO.: 1 or an amino acid sequence identical to SEQ ID NO.: 1.
3. The polypeptide sequence according to any one of items 1-2, wherein the polypeptide sequence is a neo-epitope of C4d.
4. Use of a polypeptide according to any one of items 1-3 for diagnosis of a disease.
5. Use according to item 4, wherein the disease is cancer or autoimmune diseases or conditions.
6. Use according to any one of items 4-5, wherein the diseases is SLE (serum level, deposition on platelets and erythrocytes), NSCLC (non-small cell lung cancer)—plasma and bronchioaveolar levels, antibody-mediated graft rejection (predictive in biopsies of transplants), thrombotic microangiopathy, lupus nephritis, myelopathy due to human leukaemia T-cell virus (HLTV).
7. An antibody capable of binding to a polypeptide sequence according to any one of items 1-3.
8. The antibody according to item 7, wherein the antibody is of mammalian or non-mammalian origin.
9. The antibody according to any one of items 7-8, wherein the antibody is of human, avian or murine origin.
10. The antibody according to any one of items 7-9, wherein the antibody is monoclonal or polyclonal.
11. Use of an antibody according to any one of items 7-10 for diagnosis of a disease.
12. Use according to item 11, wherein the disease is cancer or autoimmune diseases or conditions.
13. Use according to any one of items 11-12, wherein the diseases is SLE (serum level, deposition on platelets and erythrocytes), NSCLC (non-small cell lung cancer)—plasma and bronchioaveolar levels, antibody-mediated graft rejection (predictive in biopsies of transplants), thrombotic microangiopathy, lupus nephritis, myelopathy due to human leukaemia T-cell virus (HLTV).
14. A kit or kit of parts comprising an antibody according to any one of items 7-10.
15. A method of determining the level of C4d in a biological sample, the method comprising:
a) contacting the biological sample with an antibody according to any one of items 7-10, wherein the antibody is pre-loaded with radiolabelled antigen or conjugated to a suitable enzyme,
b) optionally adding a substrate capable of being digested by the enzyme conjugated to the antibody,
c) measuring the released radiolabelled antigen, or measuring the output of the digested substrate, to thereby measure the level of C4d present in the sample.

1.-15. (canceled)

16. A kit comprising:
(a) an antibody capable of recognising and binding to a polypeptide sequence comprises a sequence with at least about 98% sequence identity to SEQ ID NO.: 1, and
(b) an antibody capable of recognising and binding to a polypeptide or a fragment thereof which is conserved between the A- and B-chain in FIG. 3, wherein the antibody does not recognise or bind to SEQ ID NO.: 1.

17. The kit according to claim 16, wherein the antibody in (b) is capable of recognising an amino acid sequence with at least 4 residues of the conserved regions/amino acid sequences seen in FIG. 3.

18. The kit according to claim 16, wherein the antibodies are of mammalian or non-mammalian origin.

19. The kit according to claim 16, wherein the antibody is of human, avian or of murine origin.

20. The kit according to claim 16, wherein the antibody is monoclonal or polyclonal.

21. The kit according to claim 16, wherein the antibody in claim 16 (b) is optionally conjugated to an enzyme.

22. The kit according to claim 16, wherein the kit optionally further comprises a secondary antibody capable of binding to the Fc region of the antibody in (b), and wherein the second antibody is conjugated to an enzyme.

23. The kit according to claim 16, wherein the composition or kit of parts further comprises a substrate capable of being digested by the enzyme conjugated to the antibody.

24. The kit according to claim 16, wherein the substrate enables detection by any suitable means after being digested by the enzyme.

25. Use of a kit according to claim 16, in a diagnostic method or for diagnosis of a disease and/or condition or for measurement of complement system activation and/or screening of drugs or drug candidates.

26. Use according to claim 25, wherein the disease or condition is cancer or autoimmune diseases or conditions.

27. Use according to any one of claim 26, wherein the diseases or condition is SLE (serum level, deposition on platelets and erythrocytes), NSCLC (non-small cell lung cancer)—plasma and bronchioaveolar levels, antibody-mediated graft rejection (predictive in biopsies of transplants), thrombotic microangiopathy, lupus nephritis, myelopathy due to human leukaemia T-cell virus (HLTV).

28. A method for determining the level of C4d in a biological sample, the method comprising the steps of:
(i) providing a biological sample,
(ii) contacting the biological sample with an antibody capable of recognising and binding to a polypeptide sequence comprises a sequence with at least about 98% sequence identity to SEQ ID NO.: 1 optionally bound to a solid support,
(iii) adding to the sample obtained in (ii) an antibody capable of recognising and binding to a polypeptide or a fragment thereof which is conserved between the A- and B-chain in FIG. 3, wherein the antibody does not recognise or bind to SEQ ID NO.: 1,
(iv) optionally adding to the sample obtained in (iii) a secondary antibody capable of binding to the Fc region of an antibody capable of recognising and binding to a polypeptide or a fragment thereof which is conserved between the A- and B-chain in FIG. 3, wherein the antibody does not recognise or bind to SEQ ID NO.: 1, wherein the secondary antibody is conjugated to an enzyme,
(v) adding a substrate to the sample obtained in (iv) which is capable of being digested by the enzyme conjugated to the secondary antibody, and
(vi) measuring the output of the digested substrate.


SEQUENCE LISTING (3 sequences)

SEQ ID NO: 1 - protein, 9 aa, human
NVTLSSTGR

SEQ ID NO: 2 - protein, 380 aa, human
TLEIPGNSDPNMIPDGDFNSYVRVTASDPLDTLGSEGALSPGGVASLLRLPRGCGEQTMIYLAPTLAASRYLDKTEQWSTLPPETKDHAVDLIQKGYMRIQQFRKADGSYAAWLSRDSSTWLTAFVLKVLSLAQEQVGGSPEKLQETSNWLLSQQQADGSFQDPCPVLDRSMQGGLVGNDETVALTAFVTIALHHGLAVFQDEGAEPLKQRVEASISKANSFLGEKASAGLLGAHAAAITAYALTLTKAPVDLLGVAHNNLMAMAQETGDNLYWGSVTGSQSNAVSPTPAPRNPSDPMPQAPALWIETTAYALLHLLLHEGKAEMADQASAWLTRQGSFQGGFRSTQDTVIALDALSAYWIASHTTEERGLNVTLSSTGR

SEQ ID NO: 3 - protein, 380 aa, human
TLEIPGNSDPNMIPDGDFNSYVRVTASDPLDTLGSEGALSPGGVASLLRLPRGCGEQTMIYLAPTLAASRYLDKTEQWSTLPPETKDHAVDLIQKGYMRIQQFRKADGSYAAWLSRGSSTWLTAFVLKVLSLAQEQVGGSPEKLQETSNWLLSQQQADGSFQDLSPVIHRSMQGGLVGNDETVALTAFVTIALHHGLAVFQDEGAEPLKQRVEASISKASSFLGEKASAGLLGAHAAAITAYALTLTKAPADLRGVAHNNLMAMAQETGDNLYWGSVTGSQSNAVSPTPAPRNPSDPMPQAPALWIETTAYALLHLLLHEGKAEMADQAAAWLTRQGSFQGGFRSTQDTVIALDALSAYWIASHTTEERGLNVTLSSTGR